(19) United States
(12) Patent Application Publication
(10) Pub. No.: US 2023/0225997 A1
(43) Pub. Date: Jul. 20, 2023

(54) METHODS OF ADMINISTERING SOTALOL HYDROCHLORIDE

(71) Applicant: AltaThera Pharmaceuticals LLC, Chicago, IL (US)

(72) Inventor: Brandon Ira Kashfian, Chicago, IL (US)

(21) Appl. No.: 18/126,561

(22) Filed: Mar. 27, 2023

Related U.S. Application Data
(63) Continuation-in-part of application No. 17/585,190, filed on Jan. 26, 2022.
Application No. 17/585,190 is a continuation of application No. 16/863,567, filed on Apr. 30, 2020, now abandoned.
Application No. 16/863,567 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 16/863,567 is a continuation-in-part of application No. 16/693,310, filed on Nov. 24, 2019.
Application No. 16/693,310 is a continuation-in-part of application No. 16/103,815, filed on Aug. 14, 2018, now Pat. No. 10,512,620.
Application No. 16/103,815 is a continuation-in-part of application No. 17/306,490, filed on May 3, 2021.
Application No. 17/306,490 is a continuation of application No. 16/849,099, filed on Apr. 15, 2020, now abandoned.
Application No. 17/306,490 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 16/849,099 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 17/306,490 is a continuation-in-part of application No. 16/693,310, filed on Nov. 24, 2019.
Application No. 16/693,310 is a continuation-in-part of application No. 16/103,815, filed on Aug. 14, 2018, now Pat. No. 10,512,620.
(60) Provisional application No. 63/346,003, filed on May 26, 2022.
(60) Provisional application No. 63/332,882, filed on Apr. 20, 2022.
(60) Provisional application No. 63/331,467, filed on Apr. 15, 2022.
(60) Provisional application No. 63/323,836, filed on Mar. 25, 2022.
(60) Provisional application No. 63/009,511, filed on Apr. 14, 2020.
(60) Provisional application No. 62/987,832, filed on Mar. 10, 2020.

(51) Int. Cl. A61K 31/18; A61K 9/00; A61P 9/06

(52) U.S. Cl. CPC A61K 31/18; A61K 9/0019; A61P 9/06

(57) ABSTRACT

Methods of administering sotalol hydrochloride in an amount effective for treating a cardiovascular condition are described. An initial IV loading dose can be administered over a period of about one hour, followed by a first oral dose. The patient can be discharged from the medical facility providing cardiac monitoring prior to administration of additional oral doses. The IV and first oral doses can be administered in a manner such that maximum serum concentration of sotalol is reached and the patient can be discharged within about 7-8 hours.

### CROSS REFERENCE TO RELATED APPLICATIONS

The present application is a Continuation in Part (CIP) application of U.S. application Ser. No. 17/585,190, filed Jan. 26, 2022, which application is a Continuation application of U.S. application Ser. No. 16/863,567, filed Apr. 30, 2020. The '567 application relies on the disclosure of and claims priority to and the benefit of the filing date of U.S. Provisional Application No. 63/009,511, filed Apr. 14, 2020 and is a CIP application of U.S. application Ser. No. 16/693,312, filed Nov. 24, 2019, which application is a Continuation application of U.S. application Ser. No. 16/103,815, filed Aug. 14, 2018, which application issued on Dec. 24, 2019 as U.S. Pat. No. 10,512,620. The '567 application is a CIP application of U.S. application Ser. No. 16/693,310, filed Nov. 24, 2019, which application is a CIP application of U.S. application Ser. No. 16/103,815, filed Aug. 14, 2018. The present application is a CIP application of U.S. application Ser. No. 17/306,490, filed May 3, 2021, which application is a Continuation application of U.S. application Ser. No. 16/849,099, filed Apr. 15, 2020. The '099 application relies on the disclosure of and claims priority to and the benefit of the filing date of U.S. Provisional Application No. 62/987,832, filed Mar. 10, 2020. The '490 application is a CIP application of U.S. application Ser. No. 16/693,312, filed Nov. 24, 2019, which application is a Continuation application of U.S. application Ser. No. 16/103,815, filed Aug. 14, 2018, which application issued on Dec. 24, 2019 as U.S. Pat. No. 10,512,620. The '490 application is a CIP application of U.S. application Ser. No. 16/693,310, filed Nov. 24, 2019, which application is a CIP application of the '815 application. The present application further relies on the disclosure of and claims priority to and the benefit of the filing date of each of U.S. Provisional Application Nos. 63/346,003, filed May 26, 2022, 63/332,882, filed Apr. 20, 2022, 63/331,467, filed Apr. 15, 2022, and 63/323,836, filed Mar. 25, 2022. The disclosures of these applications are hereby incorporated by reference herein in their entireties.

### BACKGROUND OF THE INVENTION

### Field of the Invention

The present invention relates to the field of cardiovascular pharmaceutics, more particularly compositions and methods for the treatment of cardiovascular conditions, such as arrhythmias, with intravenous anti-arrhythmics, such as sotalol hydrochloride.

### Description of Related Art

Sotalol hydrochloride is a racemic mixture of d- and 1-sotalol hydrochloride. Sotalol hydrochloride is a white, crystalline solid with a molecular weight of 308.8. It is hydrophilic, soluble in water, propylene glycol, and ethanol. Chemically, sotalol hydrochloride is a d,l-N-[4-[1-hydroxy-2-[(1-methylethyl)amino]ethyl]phenyl] methane-sulfonamide monohydrochloride. (HIGHLIGHTS OF PRESCRIBING INFORMATION: Sotalol hydrochloride injection for intravenous use, Revised 7/2009). The molecular formula is C12H2ON203S·HCl and is represented by the following structural formula:

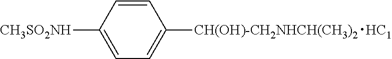

Intravenous sotalol is supplied as a sterile, clear solution in 10 ml vials, each vial containing 150 mg sotalol hydrochloride in acetate buffer. Sotalol hydrochloride is an antiarrhythmic drug with both beta adrenoreceptor blocking (Class II) and cardiac action potential duration prolongation (Class III) properties. The drug is hemodynamically well-tolerated and has a small risk of proarrhythmia. Both isomers have similar Class III activity, while the 1-isomer is responsible for virtually all of the beta blocking activity. Sotalol does not have partial beta agonist or membrane stabilizing activity (Na channel inhibition). Sotalol does not undergo metabolism and is nearly 100% bioavailable when taken on an empty stomach. Sotalol hydrochloride does not bind to plasma proteins. The pharmacokinetics of d- and 1-sotalol enantiomers are essentially identical. Excretion is predominantly via the kidney in the unchanged form and therefore lower doses are necessary in patients with renal impairment.

Sotalol is FDA approved for the maintenance of normal sinus rhythm in patients with history of highly symptomatic atrial fibrillation/flutter and for the treatment of documented life-threatening ventricular arrhythmias. Indications for sotalol include but are not limited to atrial fibrillation, atrial flutter, atrial tachycardia, focal atrial tachycardia, ventricular tachycardia, hemodynamically stable or unstable ventricular tachycardia, paroxysmal atrial fibrillation, ventricular fibrillation, supraventricular tachycardia, atrioventricular re-entrant tachycardia, atrioventricular nodal re-entrant tachycardia, paroxysmal supraventricular tachycardia, junctional ectopic tachycardia, junctional tachycardia, heart failure, coronary artery disease, pulmonary artery hypertension, patients receiving or having received extracorporeal membrane oxygenation (ECMO) therapy, or any combination thereof Sotalol is currently approved in the US for oral administration (for example, under the brand name BETAPACE AF®, Bayer HealthCare Pharmaceuticals Inc.) and is approved for IV administration (AltaThera Pharmaceuticals LLC). Sotalol is an effective antiarrhythmic agent but also can cause serious ventricular arrhythmias, primarily Torsade de Pointes (TdP) type ventricular tachycardia, a polymorphic ventricular tachycardia associated with QTc prolongation. QTc prolongation is directly related to the plasma concentration of sotalol. Steady-state plasma levels of sotalol and maximum QTc prolongation are obtained in 3 days with oral administration (i.e. after 5-6 doses when administered twice daily). QTc changes are directly related to maximum serum concentration of the sotalol. To minimize the risk of sotalol caused arrhythmias, the product label mandates that patients initiated or re-initiated on sotalol should be hospitalized for at least three days or until steady state drug levels are achieved, in a facility that can provide cardiac resuscitation and continuous electrocardiographic monitoring.

Hospitalization represents a high-cost burden in the United States. The three-day hospital stay is also burdensome to the patient, resulting in loss of productivity, time away from family, and an increased risk of hospital-acquired infections, as well as a diversion of hospital resources, staff, and patient care beds. The availability of IV sotalol offers an opportunity to improve on the sotalol loading protocol and decreases the length of hospital stay.

### SUMMARY OF THE INVENTION

Sotalol is a commonly used oral anti-arrhythmic drug for management of atrial arrhythmias. Due to a risk of pro-arrhythmia inpatient initiation of sotalol with QTc monitoring for 5 doses is a standard treatment. Patent literature relating to sotalol includes: U.S. Pat. Nos. 11,610,660, 11,583,216, 11,344,518, 10,799,138 and 10,512,620; and US Published Patent Application Nos. 2023/0075398, 2022/0339130, 2022/0241225, 2022/0142954, 2021/0283049, 2021/0076959, 2020/0338027, 2020/0253903, 2020/0093759, 2020/0085771, 2019/0380605 and 2019/0307343, which are incorporated by reference herein in their entireties. This disclosure provides a change from the guidelines for sotalol in-hospital loading that achieves steady-state blood level and maximum QTc prolongation within one day. Pharmacokinetic/pharmacodynamics (PK/PD) simulations indicate that administering an intravenous loading dose of sotalol can achieve steady state Cₘₐₓ and thus maximum QTc prolongation on Day 1. This enables physicians to evaluate the major safety concern, excessive QTc prolongation, in one day instead of 3 days.

Included in embodiments of the invention is Aspect 1, which is a method of administering sotalol hydrochloride therapy using a single intravenous (IV) dosage, the method comprising: (1) identifying a subject as having symptomatic atrial fibrillation or atrial flutter, and currently in sinus rhythm or as having ventricular tachycardia; (2) administering a single IV dosage of sotalol hydrochloride, for a period of 1 hour, to the subject who is also in a facility capable of providing cardiac resuscitation and continuous electrocardiographic monitoring, wherein the single IV dosage is administered: (a) in an amount of about 49.5-128 mg, for the subject who is naive to sotalol or who has not received sotalol for at least five (5) half-lives of sotalol; (b) in an amount of about 73.8-105.6 mg, for the subject who had received 80 mg of oral sotalol hydrochloride prior to the IV dosage; or (c) in an amount of about 88-140.8 mg, for the subject who had received 120 mg of oral sotalol hydrochloride prior to the IV dosage; (3) starting at least about 1-2 hours after completion of the single IV dosage, administering an oral dosage of sotalol hydrochloride (or optionally a corresponding IV dosage) selected from: (a) 80 mg or 120 mg, such as after completion of the (2)(a) IV dosage; or (b) 120 mg, such as after completion of the (2)(b) IV dosage; (c) 160 mg, such as after completion of the (2)(c) IV dosage; or (d) 240 mg and (4) optionally repeating oral administration of sotalol hydrochloride at least once at a selected interval optionally after the subject is discharged from the facility capable of providing cardiac resuscitation and continuous electrocardiographic monitoring.

Aspect 2 is a method, comprising: administering sotalol hydrochloride to a patient; wherein the sotalol hydrochloride is administered: as a loading dose, intravenously, over a period of about 1 hour and in an amount of 49.5 mg to 140.8 mg or about 120 mg to 260 mg; and as a first oral dose, in an amount of 80 mg, 120 mg, 160 mg, or 240 mg.

Aspect 3 is a method of initiating or escalating sotalol hydrochloride treatment in a patient comprising: (A) determining a creatinine clearance of the patient; (B) determining a QTc interval of the patient; (C) administering to the patient an IV loading dose of sotalol hydrochloride, wherein the IV loading dose is selected from an amount ranging from 49.5 mg to 140.8 mg or about 120 mg to 260 mg, and wherein optionally the IV loading dose is administered over about 60 minutes, optionally by way of several IV doses; (D) determining a second QTc interval of the patient; (E) administering one or more oral dose(s) of the sotalol hydrochloride (or optionally an IV dosage) to the patient beginning about 0.5-4 hours after completion of the administering of the IV loading dose; (F) optionally determining one or more subsequent QTc interval of the patient between one or more of the oral doses; and (G) optionally administering one or more subsequent oral doses to the patient every 12 to 48 hours, wherein the oral dose administered is selected from 80 mg, 120 mg, 160 mg or 240 mg.

Aspect 4 is a method of initiating or escalating sotalol hydrochloride treatment in a patient having symptomatic atrial fibrillation or atrial flutter, and currently in sinus rhythm, or as having ventricular tachycardia, comprising: (A) determining a creatinine clearance of the patient; (B) determining a QTc interval of the patient; (C) administering to the patient an IV loading dose of sotalol hydrochloride, wherein the IV loading dose is chosen from an amount ranging from: i. 49.5 mg to 105.6 mg, wherein the creatinine clearance of the patient is >90 mL/min; ii. 73.8 mg to 140.8 mg, wherein the creatinine clearance of the patient is >60 mL/min to 90 mL/min; or iii. 73.8 mg to 140.8 mg, wherein the creatinine clearance of the patient is 10 mL/min to 60 mL/min; (D) determining a second QTc interval of the patient; (E) administering an oral dose of the sotalol hydrochloride (or optionally an IV dosage) to the patient after completion of the administering of the IV loading dose; and (F) optionally determining one or more subsequent QTc interval of the patient after administration of the oral dose.

Aspect 4A is a method of initiating or escalating sotalol hydrochloride treatment in a patient having symptomatic atrial fibrillation or atrial flutter, and currently in sinus rhythm, or as having ventricular tachycardia, comprising: (A) determining a creatinine clearance of the patient; (B) determining a QTc interval of the patient; (C) administering to the patient an IV loading dose of sotalol hydrochloride, wherein the IV loading dose is chosen from an amount ranging from: i. 55-63 mg, such as 60 mg, wherein the patient is being initiated for a target oral dose of 80 mg, and the creatinine clearance of the patient is >90 mL/min; ii. 73.8 mg to 88 mg, such as 75 mg or 82.5 mg, wherein the patient is being initiated for a target oral dose of 80 mg, and the creatinine clearance of the patient is 10 mL/min to 90 mL/min; iii. 70-80 mg, such as 75 mg, wherein the patient is being is being escalated to a target oral dose of 120 mg, and the creatinine clearance of the patient is >90 mL/min; iv. 82-88 mg, such as 82.5 mg, wherein the patient is being is being escalated to a target oral dose of 120 mg, and the creatinine clearance of the patient is10 mL/min to 90 mL/min; v. 82 mg to 96 mg, such as 90 mg, wherein the patient is being initiated for a target oral dose of 120 mg, or is being escalated to a target oral dose of 160 mg, and the creatinine clearance of the patient is >90 mL/min; or vi. 99 mg to 140.8 mg, such as 105 mg, 112.5 mg or 125 mg, wherein the patient is being initiated for a target oral dose of 120 mg, or is being escalated to a target oral dose of 160 mg, and the creatinine clearance of the patient is 10 to 90 mL/min; vii. 120 mg to 260 mg, such as 125 mg, 140 mg, 160 mg, 180 mg, 200 mg, 220 mg, 240 mg or 260 mg, wherein the patient is being initiated for a target oral dose of 240 mg, or is being escalated to a target oral dose of 240 mg, and the creatinine clearance of the patient is 10 to 90 mL/min or >90 mL/min; (D) determining a second QTc interval of the patient; (E) administering an oral dose (or optionally an IV dosage) of the sotalol hydrochloride to the patient after completion of the administering of the IV loading dose; and (F) optionally determining one or more subsequent QTc interval of the patient after administration of the oral dose.

Aspect 5 is the method of any of Aspects 1-4, further comprising administering a second oral dose of sotalol hydrochloride to the patient 12-48 hours after the first oral dose.

Aspect 6 is the method of any of Aspects 1-5, further comprising administering one or more subsequent oral dose(s) of sotalol hydrochloride to the patient at an interval of 12-48 hours.

Aspect 7 is the method of any of Aspects 1-6, wherein the first oral dose (or a subsequent IV dose) is administered about 0.5-4 hours after completion of the administration of the IV loading dose.

Aspect 8 is the method of any of Aspects 1-7, wherein the IV loading dose is administered in an amount of about 55 mg to 73.8 mg.

Aspect 9 is the method of any of Aspects 1-8, wherein the IV loading dose is administered in an amount of about 70 mg to 88 mg.

Aspect 10 is the method of any of Aspects 1-9, wherein the IV loading dose is administered in an amount of about 80 mg to 105.6 mg.

Aspect 11 is the method of any of claims 1-10, wherein the IV loading dose is administered in an amount of 99 mg to 140.8 mg.

Aspect 12 is the method of any of Aspects 1-11, wherein the patient had received a prior dose of sotalol hydrochloride 12-24 hours prior to administration of the IV loading dose.

Aspect 13 is the method of any of Aspects 1-12, wherein the patient is capable of experiencing a sotalol hydrochloride Cmx steady state within about 8 hours of administration of the IV loading dose.

Aspect 14 is the method of any of Aspects 1-13, wherein the IV loading dose and the first oral dose are administered to the patient in a facility capable of providing cardiac resuscitation and continuous EKG monitoring.

Aspect 15 is the method of any of Aspects 1-14, wherein the second oral dose and/or subsequent oral dose(s) are administered after the patient is discharged from the facility capable of providing cardiac resuscitation and continuous EKG monitoring.

Aspect 16 is the method of any of Aspects 1-15, wherein the patient has a creatinine clearance of >90 mL/min.

Aspect 17 is the method of any of Aspects 1-16, wherein the patient has a creatinine clearance of >60 mL/min to 90 mL/min.

Aspect 18 is the method of any of Aspects 1-17, wherein the patient has a creatinine clearance of >30 mL/min to 60 mL/min.

Aspect 19 is the method of any of Aspects 1-18, wherein the patient has a creatinine clearance of 10 mL/min to 30 mL/min.

Aspect 20 is the method of any of Aspects 1-19, further comprising measuring a QT or QTc interval of the patient before administration of the IV loading dose.

Aspect 21 is the method of any of Aspects 1-20, further comprising measuring a QT or QTc interval of the patient after administration of the IV loading dose, but before administration of the first oral dose.

Aspect 22 is the method of any of Aspects 1-21, further comprising measuring a QT or QTc interval of the patient after administration of the second oral dose.

Aspect 23 is the method of any of Aspects 1-22, wherein the patient has a cardiovascular condition.

Aspect 24 is the method of any of Aspects 1-23, wherein the cardiovascular condition is selected from atrial fibrillation, atrial flutter, atrial tachycardia, focal atrial tachycardia, ventricular tachycardia, hemodynamically stable or unstable ventricular tachycardia, paroxysmal atrial fibrillation, ventricular fibrillation, supraventricular tachycardia, atrioventricular re-entrant tachycardia, atrioventricular nodal re-entrant tachycardia, paroxysmal supraventricular tachycardia, junctional ectopic tachycardia, junctional tachycardia, heart failure, coronary artery disease, and pulmonary artery hypertension, or any combination thereof.

Aspect 25 is method of any of Aspects 1-24, wherein the first oral dose amount is based on one or more QT or QTc interval.

Aspect 26 is the method of any of Aspects 1-25, wherein the second oral dose amount is different from the first oral dose amount.

Aspect 27 is the method of any of Aspects 1-26, wherein the patient is being treated for atrial fibrillation and/or atrial flutter.

Aspect 28 is the method of any of Aspects 1-27, wherein the patient is or is not currently in normal sinus rhythm.

Aspect 29 is a method of administering sotalol hydrochloride therapy using a single intravenous (IV) dosage, the method comprising: (1) administering to a subject a single IV dosage of sotalol hydrochloride, for a period of about 1 hour, wherein the single IV dosage is administered: (a) in an amount of about 49.5-128 mg, e.g., for the subject who is naive to sotalol or who has not received sotalol for at least five (5) half-lives of sotalol; (b) in an amount of about 73.8-105.6 mg, e.g., for the subject who had received 80 mg of oral sotalol hydrochloride prior to the IV dosage; or (c) in an amount of about 88-141 mg, e.g., for the subject who had received 120 mg of oral sotalol hydrochloride prior to the IV dosage; or (d) in an amount of about 140-260 mg, e.g., for the subject who had received 160 mg of oral sotalol hydrochloride prior to the IV dosage; or (e) in an amount of about 90-260 mg, e.g., for the subject who is being initiated for a 160 mg or 240 mg oral sotalol hydrochloride dose and (2) starting at least about 1 hour (e.g. 1-8 hours) after completion of the single IV dosage, administering an oral dosage of sotalol hydrochloride selected from: (a) 80 mg or 120 mg, such as after completion of the (1)(a) IV dosage; or (b) 120 mg, such as after completion of the (1)(b) IV dosage; (c) 160 mg, such as after completion of the (1)(c) IV dosage; or (d) 240 mg, such as after completion of the (1)(d) IV dosage; or (e) 160 mg or 240 mg, such as after completion of the (1)(e) IV dosage.

Aspect 30 is the method of any of Aspects 1-29, wherein: the single IV dosage and the oral dosage are administered to the subject while admitted to a facility capable of providing cardiac resuscitation and continuous electrographic monitoring; or the single IV dosage is administered to the subject while admitted to a facility capable of providing cardiac resuscitation and continuous electrographic monitoring, then the subject is released from the facility before any oral dosages of sotalol hydrochloride are administered; optionally, wherein the oral dosage is administered about 1-4 hours after completion of the administration of the IV loading dose.

Aspect 31 is the method of any of Aspects 1-30, wherein: the single IV dosage and the oral dosage are administered to the subject while admitted to a facility capable of providing cardiac resuscitation and continuous electrographic monitoring; the subject is released from the facility capable of providing cardiac resuscitation and continuous electrographic monitoring; and the subject is administered one or more additional oral dosage of sotalol hydrochloride.

Aspect 32 is the method of any of Aspects 1-31, wherein the oral dosage is administered about 1-4 hours after completion of the administration of the IV loading dose.

Aspect 33 is the method of any of Aspects 1-32, wherein the subject experiences or is expected to experience a sotalol hydrochloride Cₘₐₓ steady state within about 8 hours of the administering of the single IV dose.

Aspect 34 is the method any of Aspects 1-33, wherein the subject has a cardiovascular condition selected from atrial fibrillation, atrial flutter, atrial tachycardia, focal atrial tachycardia, ventricular tachycardia, hemodynamically stable or unstable ventricular tachycardia, paroxysmal atrial fibrillation, ventricular fibrillation, supraventricular tachycardia, atrioventricular re-entrant tachycardia, atrioventricular nodal re-entrant tachycardia, paroxysmal supraventricular tachycardia, junctional ectopic tachycardia, junctional tachycardia, heart failure, coronary artery disease, and pulmonary artery hypertension, or any combination thereof.

Aspect 35 is the method of any of Aspects 1-34, wherein the subject is being treated for atrial fibrillation and/or atrial flutter and is currently in normal sinus rhythm.

Aspect 36 is the method of any of Aspects 1-35, wherein: the single IV dosage is in an amount of 49.5-128 mg; the oral dosage is 80 mg or 120 mg; and the subject is naive to sotalol or has not received sotalol for at least five (5) half-lives of sotalol.

Aspect 37 is the method of any of Aspects 1-36, wherein: the single IV dosage is in an amount of 73.8-105.6 mg; the oral dosage is 120 mg; and the subject had received 80 mg of oral sotalol hydrochloride prior to the IV dosage.

Aspect 38 is the method of any of Aspects 1-37, further comprising: determining a first QTc interval of the patient is less than 450 ms; and determining a second QTc interval of the patient is less than 500 ms and is less than a 20% increase from the first QTc interval.

Aspect 39 is a method of initiating or escalating sotalol hydrochloride treatment in a patient, comprising: (A) determining a creatinine clearance of the patient is >60 mL/min; (B) determining a first QTc interval of the patient is 450 ms or less; (C) administering to the patient an IV loading dose of sotalol hydrochloride, wherein the IV loading dose is chosen from an amount ranging from: i. 55-88 mg, wherein the patient is being initiated for a target oral dose of 80 mg; ii. 70-88 mg, wherein the patient is being is being escalated to a target oral dose of 120 mg; iii. 82 mg to 140.8 mg, wherein the patient is being initiated for a target oral dose of 120 mg, or is being escalated to a target oral dose of 160 mg or 240 mg; (D) determining a second QTc interval of the patient is less than 500 ms and is less than a 20% increase from the first QTc interval; and (E) administering a first oral dose of the sotalol hydrochloride to the patient after completion of the administering of the IV loading dose.

Aspect 40 is the method of any of Aspects 1-39, wherein the first oral dose is administered at least about 1 hour after completion of the administration of the IV loading dose.

Aspect 41 is the method of any of Aspects 1-40, wherein the patient experiences or is expected to experience a sotalol hydrochloride Cₘₐₓ steady state within about 8 hours of the administering of the single IV dose.

Aspect 42 is the method of any of Aspects 1-41, wherein the patient has a cardiovascular condition selected from atrial fibrillation, atrial flutter, atrial tachycardia, focal atrial tachycardia, ventricular tachycardia, hemodynamically stable or unstable ventricular tachycardia, paroxysmal atrial fibrillation, ventricular fibrillation, supraventricular tachycardia, atrioventricular re-entrant tachycardia, atrioventricular nodal re-entrant tachycardia, paroxysmal supraventricular tachycardia, junctional ectopic tachycardia, junctional tachycardia, heart failure, coronary artery disease, and pulmonary artery hypertension, or any combination thereof.

Aspect 43 is the method of any of Aspects 1-42, wherein the patient is being treated for atrial fibrillation and/or atrial flutter and is currently in normal sinus rhythm.

Aspect 44 is the method of any of Aspects 1-43, wherein: the patient is being initiated for a target oral dose of 80 mg; the creatinine clearance of the patient is >90 mL/min; and the IV loading dose is administered in an amount of 55-63 mg.

Aspect 45 is the method of any of Aspects 1-44, wherein: the patient is being initiated for a target oral dose of 120 mg; the creatinine clearance of the patient is >90 mL/min; and the IV loading dose is administered in an amount of 82 mg to 96 mg.

Aspect 46 is the method of any of Aspects 1-45, wherein: the patient is being escalated to a target oral dose of 120 mg; the creatinine clearance of the patient is >90 mL/min; and the IV loading dose is administered in an amount of 70 mg to 80 mg.

Aspect 47 is the method of any of Aspects 1-46, wherein at least one oral dose amount is based on the first QTc interval and/or the second QTc interval.

Aspect 48 is the method of any of Aspects 1-47, wherein: the subject has a cardiovascular condition selected from atrial fibrillation, atrial flutter, or ventricular tachycardia; the oral dosage is administered about 1-4 hours after completion of the administration of the IV loading dose; and there is less than a 20% increase in a change in QTc interval of the subject after administering the IV dosage.

### DETAILED DESCRIPTION OF VARIOUS EMBODIMENTS OF THE INVENTION

Reference will now be made in detail to various illustrative implementations. It is to be understood that the following discussion of implementations is not intended to be limiting.

AF is atrial fibrillation.

AFL is atrial flutter.

AF/AFL is atrial fibrillation and/or atrial flutter.

IV is intravenous.

PO means “per os” and refers to an oral dosing regimen.

BID means “bis in die” and means twice a day.

QD means “quaque die” and means once a day.

QID means “quater in die” and means four times a day.

Patient (or subject) refers to a human patient.

BP is blood pressure.

HR is heart rate.

Renally impaired refers to patients having creatinine clearance rates of 60 mL/min, such as 30 mL/min.

Escalation means increasing the sotalol dosage of a patient already receiving sotalol, for example where a subject is currently taking a specific amount of an oral dose and escalation involves administering one or more IV doses to escalate the subject to a higher target oral dose.

Sotalol and sotalol hydrochloride (used interchangeably herein) refer to d,l-sotalol hydrochloride.

The terms “treat,” “treating,” and “treatment” refer to any indicia of success in the treatment or amelioration of an injury, disease, or condition, including any objective or subjective parameter such as abatement; remission; diminishing of symptoms or making the injury disease, or condition more tolerable to the subject; slowing in the rate of degeneration or decline; or improving a subject's physical or mental well-being. The treatment or amelioration of symptoms can be based on objective or subjective parameters, including the results of a physical examination, neuropsychiatric examinations, or psychiatric evaluation.

Cₘₐₓ ss is the maximal concentration obtained at steady state.

QT is the interval measured from the start of the Q wave or QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by mathematical correlation of the QT interval and the heart rate.

ΔQTc is the difference between a QTc measurement taken prior to the start of treatment and a QTc measured after the start of treatment (e.g., during loading or maintenance).

The term “about” used herein in the context of quantitative measurements means the indicated amount ±10%. For example, “about 2 mg” can mean 1.8-2.2 mg.

IV and Oral Dosing

In embodiments, the loading dose is delivered by way of an IV infusion over a period of about 1 hour, such as in the range of about 55 minutes to 65 minutes, about 50 minutes to about 70 minutes, or about 45 minutes to about 75 minutes.

In embodiments, the loading dose is administered in an amount of about 60 mg, about 75 mg, or about 90 mg, such as about 55 mg to about 65 mg, about 50 mg to about 70 mg, about 70 mg to about 80 mg, about 65 mg to about 85 mg, about 85 mg to about 95 mg, about 80 mg to about 100 mg, or about 45 mg to about 105 mg, or about 110-128 mg, or about 99-140.8 mg or about 49.5-70.2 mg, or about 73.8-105.6 mg, or about 63-88.2 mg, or about 55-64 mg, or about 82-96 mg, or about 49.5-70.4 mg, or about 73.8-105.6 mg, or about 63-88 mg, or about 70-80 mg, or any range in between. The loading dose can also be expressed in mg/min., such as from about 0.825-1.17 mg/min. (49.5-70.2 mg), or from about 1.23-1.76 mg/min. (73.8-105.6 mg), or from about 1.65-2.35 mg/min. (99-141 mg), or from about 1.05-1.47 mg/min. (66-88.2 mg), or any range in between.

In embodiments, one or more first oral dose is administered and a first oral dose is administered about 4 hours after the conclusion of the administration of the loading dose, such as about 3.75 hours to about 4.25 hours, about 3.5 hours to about 4.5 hours, about 3.25 hours to about 4.75 hours, or about 3 hours to about 5 hours after administration of the loading dose. In embodiments, the subject/patient is administered the oral dose after being released from the hospital and not under hospital monitoring.

In embodiments, the one or more oral dose is selected from about 80 mg, about 120 mg, or about 160 mg or about 240 mg.

In embodiments, the oral dose is selected based on a patient's creatinine clearance. For example, in some cases a patient/subject with a lower creatinine clearance may receive a lower dose and a patient with a higher creatinine clearance may receive a higher dose, or in some cases a patient/subject with a higher creatinine clearance may receive a lower dose and a patient with a lower creatinine clearance may receive a higher dose. In some cases, the amount of sotalol hydrochloride appropriate for a subject with a CrCl of 10-30 mL/min or 30-60 mL/min can be higher than is appropriate for a subject with a CrCl of >90 mL/min.

In embodiments, the oral dose is selected based on a patient's change in QTc (for example, a patient that experiences an increase in QTc of less than 20% after receiving the loading dose may receive a higher oral dose than a patient that experiences an increase in QTc of 20% or greater after receiving the loading dose).

In embodiments, subsequent oral doses are given after the first oral dose. In embodiments, the subsequent oral doses are administered at intervals of about 12 hours, about 24 hours, or about 48 hours. In embodiments, the subject/patient is administered subsequent oral dose(s) after being released from the hospital or other facility capable of providing cardiac resuscitation and continuous electrocardiographic monitoring.

In embodiments, the IV dose and first oral dose are administered in a hospital or other facility that can provide cardiac resuscitation and continuous electrocardiographic monitoring and subsequent oral doses are administered by the patient after discharge from the facility/hospital.

In embodiments, a single IV dosage and the oral dosage are administered to the subject while admitted to a facility capable of providing cardiac resuscitation and continuous electrographic monitoring; or the single IV dosage is administered to the subject while admitted to a facility capable of providing cardiac resuscitation and continuous electrographic monitoring, then the subject is released from the facility before any oral dosages of sotalol hydrochloride are administered; optionally, wherein the oral dosage is administered about 1-4 hours after completion of the administration of the IV loading dose.

In embodiments, the loading dose and maintenance dose(s) are given in amounts and at time intervals such that the patient achieves Cₘₐₓ ss within about 6, 7 or 8 hours of the start of the administration of the loading dose.

In embodiments, the loading dose and maintenance dose(s) are given in amounts and at time intervals such that the patient experiences or is expected to experience steady state blood levels and max QTc within about 8 hours, such as within about 6 hours, of the start of the administration of the loading dose.

In embodiments, the patient receiving sotalol hydrochloride has experienced AF, but is in normal sinus rhythm prior to administration of the sotalol hydrochloride loading dose, or has experienced VT (ventricular tachycardia) and is or is not currently in sinus rhythm.

### Example 1

The safety and feasibility of the intravenous loading strategy with Sotalol achieving steady state or expecting to achieve steady state and thus maximum QTc prolongation within 7-8 hours instead of the traditional 3-day oral dose titration is evaluated. In an embodiment of the invention, the intravenous loading regimen is followed by oral sotalol administration 4 hours after the conclusion of the IV infusion (and expected to be administered every 12 hours thereafter). This loading program is designed to result in sotalol peak concentrations by the end of IV loading dose which are comparable to the steady state and maximum QTc seen on Day 3 after the last in-hospital oral dose.

From eligible patients on the morning of Day 1, blood sample for serum creatinine and electrolyte measurements can be taken, or prior samples can be used, obtained within the previous two days. Creatinine clearance can be measured using the Cockroft-Gault formula. Patients who are in atrial fibrillation will undergo direct current cardioversion (DCCV) and/or conversion using sotalol IV as indicated and at the discretion of the treating physician. Patients should preferably be in normal sinus rhythm for at least two hours post DCCV/conversion to be eligible for the 1 Day protocol, but in embodiments can be treated even if not currently in sinus rhythm.

The patient, who is in a facility capable of providing cardiac resuscitation and continuous electrographic monitoring, is placed on telemetry and a 12-lead ECG performed to measure the QTc interval. The corrected QTc interval for heart rate (QTc) is calculated by the Bazett formula. If creatinine clearance is ≥60 mL/min and the QTc interval is ≤450 ms and the patient is in sinus rhythm (or in embodiments, not in sinus rhythm), the patient is administered sotalol, with an initial IV loading dose administered over a period of up to 1 hour (e.g., 60 mg, 82.5 mg, 90 mg, 105 mg, 125 mg, 140 mg, 160 mg, 220 mg, 260 mg etc., whether by way of a single IV dose or multiple doses). If the patient's creatinine clearance is ≤60 mL/min, then the initial 1-hr IV loading dose is typically 75 mg or 112.5 mg. Four hours after the conclusion of the IV loading dose (at five (5) hours after the start of infusion), patients begin the respective oral sotalol therapy (80 or 120 mg, to be administered every 12h). Patients can be administered the first and/or subsequent oral doses in a facility capable of providing cardiac resuscitation and continuous electrographic monitoring or after being released from such facility. The choice of dosing regimen is based on the physician's judgment with respect to target oral maintenance dose. In general, the lowest effective dose should be given to each patient to avoid adverse events, primarily proarrhythmia (Torsade de Pointes). In patients with normal CrCl the starting oral dose is 120 mg given every 12 hours. The first oral dose after the IV loading dose is given 4 hours after the IV loading dose is complete. After the IV loading dose, a 12 lead ECG is done to measure the QTc change from baseline. If the QTc increase is less than 20%, the first oral dose is given (e.g., at or about hour 5). After two hours, a repeat 12 lead ECG is done (e.g., at or about hour 7). If the QTc interval remains stable, the patient is discharged home with a mobile cardiac outpatient telemetry (MCOT) unit (e.g., within about 7-8 hours after the start of the protocol. The patient is asked to capture the rhythm strip prior to the oral dose and 2.5 hours after each dose, and if the patient develops symptoms of palpitations, lightheadedness and/or shortness of breath.

An increase in QTc by 20% or greater than 500 ms should be cause to reduce or stop infusion or reduce or stop oral administration. If the QTc is prolonged >20% after the 1st oral dose, patient is admitted to the hospital and given the next lower dose at 12 hours. For example, if the oral dose is 120 mg, it can be reduced to 80 mg. QTc will be checked and if within the acceptable limits the patient is discharged home with a MCOT (or similar monitoring technology) and follow the monitoring regimen outlined above. On day 4 the patient will visit the physician's office and obtain 12 lead ECG (or similar evaluation) to assess QTc interval.

In embodiments, subjects can be observed in a hospital for the first 8 hours on telemetry and the next 4 oral doses at home with MCOT, or similar monitoring. Sotalol may be continued at the discretion of the physician.

Patients with history of highly symptomatic persistent or paroxysmal AF who are scheduled for sotalol therapy once in sinus rhythm, or patients who have experienced VT who are or are not in sinus rhythm, can be administered the 1 Day protocol.

Following DCCV of atrial fibrillation, the QT interval transiently and variably prolongs (Darbar, D. et al., A Rate-Independent Method of Assessing QT-RR Slope Following Conversion of Atrial Fibrillation. Journal of Cardiovascular Electrophysiology. 2007; 18(6):636-641). Sotalol can further augment bradycardia and QTc prolongation following DCCV (HIGHLIGHTS OF PRESCRIBING INFORMATION: Sotalol hydrochloride injection for intravenous use, Revised 7/2009). Furthermore, there is an immediate increase in QTc dispersion following DCCV that lasts approximately 15 minutes (Boriani, G. et al., Increase in QT/QTc dispersion after low energy cardioversion of chronic persistent atrial fibrillation. International Journal of Cardiology. 2004;95(2-3):245-250). These phenomena may explain the transient increase in vulnerability for proarrhythmia following DCCV. To avoid these confounding factors, a 2-hour interval without any pharmaceutical intervention should elapse before baseline QTc is measured, and then sotalol infusion may begin. If the patient presents in sinus rhythm the infusion can be started immediately after the 12 lead ECG.

TABLE 1
Intravenous-to-Oral Loading Protocol

| Selected Oral Sotalol Dose | Corresponding IV Loading Dose |
|---|---|
| (q12 h) | (infused over 1 hour) |
| 80 mg | 60 mg |
| 120 mg | 90 mg |

Intravenous-to-Oral Loading Protocol

1. The subject is admitted to the hospital following DCCV (or other conversion) for atrial fibrillation. Subjects having normal serum electrolytes, creatinine clearance >60 mL/min, and QTc ≤450 ms or JT≤330 ms if QRS is >100 ms are preferred for the 1 Day protocol.

2. Telemetry can begin and the target oral dose of sotalol selected: 80 mg or 120 mg PO every 12 h. This selection is made by the subject's treating physician. In some embodiments, the selection can be made in accordance with the dosing and creatinine clearance ranges provided in Table 7. The subject will be observed via cardiac telemetry in the hospital for 6 to 8 hours and at home via MCOT.

3. Administer a 1-hour loading infusion using a volumetric infusion pump at a dose that is dependent on the oral dose selected in Step 2. (See Table 1 above and Table 7 below)

4. Record the subject's heart rate, blood pressure, and QTc interval once at baseline (before infusion), then every 15 minutes during the loading infusion and for 30 minutes following completion of the infusion.

5. Upon the completion of infusion, flush the IV line by administering 20 mL of 5% dextrose solution.

6. Four hours after the conclusion of the IV loading dose (Five (5) hours after the start of infusion), begin the subject's oral sotalol therapy as selected in Step 2.

7. Record the subject's heart rate, blood pressure, and QTc interval just prior to administering the first oral sotalol dose, then at 30 minutes, 1 hour, 2 hours, and 2.5 hours following administration, QTc may be measured from telemetry but with 12 lead ECG at 2.5 hour after the administration of the 1st oral loading dose. See Tables 3-5 for data collection timelines.

8. A second oral dose of sotalol is given at home. Patients will continue to be monitored according to Step 9 for an additional 3 days, or more, to rule out any side effects of the drug.

9. The patient will record heart rate, blood pressure in symptom diary and perform a manual transmission with MCOT just prior to administering every future oral sotalol dose, and then again at 2.5 hours after each dose. The QTc interval is measured on the 12 lead ECG while the patient is in the hospital and using the MCOT device while they are at home.

10. Patients will continue on oral sotalol at the discretion of their treating physician and be followed as standard of care.

Dose Escalation Protocol

If the subject has been on oral sotalol chronically at 80 mg every 12h or 120 mg every 12h and in the opinion of the treating physician the dose of sotalol needs to be increased, a one-day intravenous to oral dosing escalation regimen can also be utilized to achieve steady-state and the rest of the home dosing regimen.

TABLE 2
Dose Escalation Protocol

| Oral Sotalol Dose Escalation | Corresponding IV Loading Dose |
|---|---|
| (q12 h) | (infused over 1 hour) |
| 80 mg to 120 mg | 75 mg |

1. If the physician elects to increase the oral dose of sotalol, a new target oral sotalol dose is selected: from 80 mg to 120 mg to 160 mg to 240 mg every 12h. In some embodiments, the dosing can be performed in accordance with the dosing and creatinine clearance ranges of Table 7.

2. 12 hours following the last administration of the current/lower oral dose, administer a 1-hour loading infusion using a volumetric infusion pump at a dose that is dependent on the oral dose escalation selected in Step 1. (See Table 2 above and Table 7 below.) For example, if creatinine clearance is >60 mL/min and the QTc interval is ≤450 ms and the patient is in sinus rhythm (or in embodiments, not in sinus rhythm), the patient will be administered sotalol, with an initial IV loading dose administered over a period of 1 hour (e.g., 75 mg, 82.5 mg, 90 mg or 105 mg). If creatinine clearance is ≤60 mL/min, then the initial 1-hr IV loading dose is 82.5 mg or 105 mg.

3. Starting at Step 4 in section 9.1 above, follow the Intravenous-to-Oral Loading Protocol, recording all data in the same manner at the same time points using the beginning of this escalation protocol as a new time reference.

Table 3. Data Collection Timeline—IV Dose

IV Dose

| | 0:00 | | | | | | | |
|---|---|---|---|---|---|---|---|---|
| | (pre- | 0:15 | 0:30 | 0:45 | 1:00 | 1:15 | 1:30 | 2:00 |
| Time | infusion) | minutes | minutes | minutes | hour | hours | hours | hours |
| Vitals | ✓ | ✓ | ✓ | ✓ | ✓ | ✓ | ✓ | ✓ |

TABLE 4
Data Collection Timeline-IV Dose

| | 0:00 | | | | |
|---|---|---|---|---|---|
| | (3:00 hours after the 2:00 | 0:30 | 1:00 | 2:00 | 2:30 |
| Time | hour time point above) | minutes | hour | hours | hours |
| Vitals | ✓ | ✓ | ✓ | ✓ | ✓ |

TABLE 5
Data Collection Timeline-Oral Dose
Future Oral Doses

| | 0:00 | 2:30 |
|---|---|---|
| Time | (pre-dose) | hours |
| Vitals | ✓ | ✓ |

Precautions

Intravenous sotalol may result in bradycardia and/or hypotension and can result in proarrhythmia. Patient vital signs and ECG monitoring should be performed. Patients who develop significant bradycardia, heart block or pauses that are hemodynamically or otherwise destabilizing in the opinion of the physician can be discontinued treatment at any time. For example, development of a QTc prolongation of greater than 500 ms will be cause for discontinuation. In embodiments, development of a QTc prolongation of >500 ms will be cause for discontinuation. Patients who develop proarrhythmia should be discontinued treatment.

Adverse Events

An adverse event is any untoward, undesired, unplanned clinical event in the signs, symptoms, disease, or laboratory or physiological observations occurring in a human being, regardless of causal relationship, including the following:

any clinically significant worsening of a pre-existing condition;

an adverse event occurring from overdose, whether accidental or intentional;

an adverse event that has been associated with the discontinuation of the drug.

Any symptomatic or clinically significant arrhythmia or bradyarrhythmias including Torsades de Pointes (TdP) and sustained VT will be considered an SAE.

Adverse events which cause any of the following:

results in death;

is life-threatening;

requires inpatient hospitalization or causes prolongation of existing hospitalization;

results in persistent or significant disability/incapacity;

requires intervention to prevent permanent impairment or damage

are considered a serious adverse event (SAE).

Risks/Benefits

The following are minimal anticipated risk for the use of both IV and oral Sotalol:

Arrhythmias

Bradycardia

Shortness of Air

Fatigue

All patients should be monitored while in hospital on continuous telemetry and while at home with a mobile cardiac outpatient telemetry. The patient's blood pressure and heart rate should be monitored and 12-lead ECGs completed at various times.

It is expected that according to the 1 Day protocol, an intravenous loading regimen followed by q12h oral sotalol administration will result in sotalol concentration and QTc peaks on day 1 (IV to oral loading) which are comparable to the steady-state and maximum QTc seen on Day 3 (steady-state).

Results

In one study, patients with atrial fibrillation and atrial flutter undergoing a rhythm control strategy with sotalol were administered the 1 Day protocol. All patients underwent cardioversion for accurate assessment of QTc while in sinus rhythm. Dose of IV sotalol was calculated based on target oral dose as indicated by baseline QTc interval and renal function. (CrCl >90 ml/min-80 mg oral dose=60 mg IV; 120 mg oral dose=90 mg IV; CrCl 60-89 ml/min, 80 mg oral dose=82.5 mg IV and 120 mg PO was equivalent to 125 mg IV). Patients heart rate (HR), blood pressure (BP), QTc were measured via EKG at baseline then every 15 minutes during the loading infusion and at 30 minutes and 2 hours post completion. Patients were started on oral therapy after 4 hours post infusion HR, BP and QTc interval were obtained 2 hours after the 1^{st} oral dose. Patients were monitored on telemetry for 6-8 hours post IV loading infusion and discharged with a mobile cardiac outpatient telemonitor (MCOT). Initiating was considered complete after the second oral dose at home. Patients were instructed to record HR, BP in diary and perform a manual transmission with MCOT prior to dose of sotalol then again at 2 hours after each dose. Patients were monitored via MCOT for additional 3 days.

Safety endpoints can include: 1) Severe bradycardia (30% decrease from baseline or HR≤40 bpm); 2) 15% change in QTc interval from baseline or >500 msec; 3) Incidence of ventricular arrhythmias including TdP. Pre-specified secondary endpoints can include: 1) Change in QTc interval from baseline to the 4th oral dose; 2) Incidence of conduction system abnormalities including sinus dysfunction, AV block, bundle branch blocks and ventricular arrhythmias.

In another study, a total of 120 patients with AF/AFL were enrolled for sotalol initiation between 2021 and 2022 who met the study inclusion criteria. The study group was compared to a cohort of 120 patients who were admitted to the hospital for PO sotalol loading during the same time period at the same centers. The two groups were well balanced for baseline demographics and clinical characteristics. Prior ablation occurred in 23.300 of patients, and 25.80% had undergone cardioversion before enrollment. In the IV arm 33.300 (n=40) and 66.600 (n=80) were started on IV sotalol 120 mg and 80 mg respectively. In the PO arm the starting doses of 80 mg, 120 mg and 160 mg were given in 20.8% (n=25), 60% (n=72) and 19.1% (n=23) patients respectively. Mean age was 68.2+9.8. Baseline characteristics and medical history are described in Table 6.

TABLE 6

| | IV Sotalol | Oral Sotalol | |
|---|---|---|---|
| Variables | (n = 120) | (n = 120) | P-value |
| Age (years) | 68.2 ± 9.8 | 65.6 ± 11.2 | 0.06 |
| Male | 76 (63.3) | 73(60.8) | 0.79 |
| Body Mass Index | 33.6 ± 7.8 | 32.5 ± 8 | 0.28 |
| CHADS₂ VASC₂ Score | 2.8 ± 1.6 | 2.9 ± 1.8 | 0.63 |
| Hypertension (n, %) | 94 (78.3) | 98(81.6) | 0.52 |
| Diabetes Mellitus (n, %) | 34 (28.3) | 31(25.8) | 0.71 |
| Hyperlipidemia (n, %) | 72(60) | 82(82.3) | 0.18 |
| Coronary artery disease (n, %) | 58(48.3) | 56(46.6) | 0.80 |
| Congestive Heart Failure (n, %) | 24(20) | 22(18.3) | 0.74 |
| Obstructive sleep apnea (n, %) | 35 (29.1) | 31(25.8) | 0.56 |
| Current Smoker (n, %) | 15(12.5) | 13(10.8) | 0.59 |
| History of Previous stroke/Transient | 9 (7.5) | 8(6.7) | 0.80 |
| ischemic attack (n, %) | | | |
| Presence of implantable loop recorder | 35(29.1) | 29(24.1) | 0.38 |
| (n, %) | | | |
| Maintenance Sotalol Dose (n, %) | | | |
| 80 mg | 44(36.7) | 39(32.5) | 0.50 |
| 120 mg | 76(63.3) | 65(54.2) | 0.15 |
| 160 | — | 16(13.3) | |

### EXAMIPLE 2

In an embodiment of the invention, sotalol therapy is initiated or escalated by administering to a patient in need thereof an IV loading dose and one or more oral dose of sotalol according to the criteria shown in Table 7.

TABLE 7
IV Sotalol Loading Dosage and Initiation-Escalation Protocol

| | IV loading dose (1 h infusion) | | | | | |
|---|---|---|---|---|---|---|
| | when the oral dose is going from . . . | | | | | |
| | Sotalol | | Sotalol | | Minimum | Oral |
| Creatinine | Initiation | | Escalation | | delay to | dosing |
| Clearance* | 0 to | 0 to | 80 to | 120 to | first oral | interval |
| (mL/min) | 80 mg** | 120 mg | 120 mg | 160 mg | dose (h) | (h) |
| >90 | 60 | 90 | 75 | 90 | 4 | 12 |
| >60-90 | 82.5 | 125 | 82.5 | 105 | 4 | 12 |
| >30-60 | 75 | 112.5 | 82.5 | 105 | 6 | 24 |
| 10-30 | 75 | 112.5 | 82.5 | 105 | 12 | 48 |

*Calculated using Cockcroft-Gault formula
**Recommended starting dose

The Cockcroft-Gault formulas for creatinine clearance (CrCl) are:

CrCl (male)=((140−age)×weight in kg)/(serum creatinine x 72)

CrCl (female)=CrCl (male)×0.85

The recommended starting dose of 80 mg is the FDA recommended dosage. A physician can elect to start a patient on a higher dose (e.g., 120 mg), if deemed appropriate.

The minimum delay to first oral dose is the time from the end of the IV infusion to the first oral dose. In embodiments, the delay to the first oral dose is chosen from about 4 hours, 5 hours, 6 hours, 7 hours, 8 hours, 9 hours, 10 hours, 11 hours, or 12 hours.

The oral dosing interval refers to the time between oral dosages. 12 h is B.I.D. (or BID). 24 h is Q.D. (or QD).

In embodiments, the loading dose is delivered via IV infusion over a period of up to about 1 hour, such as in the range of about 55 minutes to 65 minutes, about 50 minutes to about 70 minutes, or about 45 minutes to about 75 minutes. In embodiments, the subject/patient is administered the loading dose while the patient is in a facility capable of providing cardiac resuscitation and continuous electrographic monitoring.

Table 7 shows that the IV loads for initiation of a target dose of 80 mg are 60 mg (>90 mL/min CrCl), 82.5 mg (60-90 mL/min CrCl), and 75 mg (30-60 mL/min CrCl and 10-30 mL/min CrCl). Additional examples of the IV load for the target dose of 80 mg include 49-90 mg, such as 55-85 mg. Further examples include 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, and 85 mg.

Table 7 shows that the IV loads for initiation of a target dose of 120 mg are 90 mg (>90 mL/min CrCl), 125 mg (60-90 mL/min CrCl), and 112.5 mg (30-60 mL/min CrCl and 10-30 mL/min CrCl). Additional examples of the IV load for the target dose of 120 mg include 75-135 mg, such as 82-128 mg. Further examples include 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, and 135 mg.

Table 7 shows that the IV loads for escalation from 80 to 120 mg are 75 mg (>90 mL/min CrCl), and 82.5 mg (60-90 mL/min CrCl, 30-60 mL/min CrCl, and 10-30 mL/min CrCl). Additional examples of the IV load for escalation to 120 mg include 63-96 mg, such as 65-90 mg. Further examples include 65, 66, 67, 68, 69, 70, 71, 72, 73, 74, 75, 76, 77, 78, 79, 80, 81, 82, 83, 84, 85, 86, 87, 88, 89, and 90 mg.

Table 7 shows that the IV loads for escalation from 120 to 160 mg are 90 mg (>90 mL/min CrCl), and 105 mg (60-90 mL/min CrCl, 30-60 mL/min CrCl, and 10-30 mL/min CrCl). Additional examples of the IV load for escalation to 160 mg include 80-120 mg or 88-140.8 mg. Further examples include 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, and 120 mg.

In embodiments, a patient can receive an IV loading dose capable of increasing the patient's oral dose from 160 mg to 240 mg or initiation of the patient at a 240 mg oral dose. For example, the IV loads for escalation from 160 mg to 240 mg or initiation for a 240 mg oral dose can be in the range of 105 mg to 260 mg, such as 120-140 mg or 110-165 mg (>90 mL/min CrCl), and 130 mg to 240 mg (60-90 mL/min CrCl, 30-60 mL/min CrCl, and 10-30 mL/min CrCl). Additional examples of the IV load for a target of 240 mg include 125-180 mg, 135-200 mg, or 160-220 mg. Further examples include 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, or 260 mg.

In embodiments, the time of when oral dosing begins depends on the CrCl of the patient. Patients can be administered the first and/or subsequent oral doses in a facility capable of providing cardiac resuscitation and continuous electrographic monitoring or after being released from such facility. Oral dosing for CrCl of >90 mL/min and 60-90 mL/min typically begins 4 h after completion of the IV infusion (e.g., 5 hours after the start of a 1 h IV). Oral dosing for CrCl of 30-60 mL/min typically begins 6 h after completion of IV infusion (e.g., 7 h after the start of a 1 h IV). Oral dosing for CrCl of 10-30 mL/min typically begins 12 h after IV infusion (e.g., 13 h after the start of a 1 h IV). Additional examples of when the oral dosing begins for a CrCl of>90 mL/min include 2-6 h after completion of infusion. Further examples include 2, 3, 4, 5, to 6 h. Additional examples of when the oral dosing begins for a CrCl of 60-90 mL/min include 2-6 h after completion of infusion. Further examples include 2, 3, 4, 5, to 6 h. Additional examples of when the oral dosing begins for a CrCl of 30-60 mL/min include 4-8 h after completion of infusion. Further examples include 4, 5, 6, 7, to 8 h. Additional examples of when the oral dosing begins for a CrCl of 10-30 mL/min include 10-14 h after completion of infusion. Further examples include 10, 11, 12, 13, to 14 h.

### Example 3

An example sotalol treatment protocol for a patient experiencing AF is described herein. The male patient, age 60, is admitted to the hospital to initiate treatment. In embodiments, the hospital is a facility capable of providing cardiac resuscitation and continuous electrographic monitoring. The patient's creatinine clearance is determined to be >90 mL/min. The patient is connected to an electrocardiograph and an initial QTc is determined to be less than 450 ms. A physician determines the patient should be initiated on an oral sotalol dosing regimen of 80 mg by mouth twice a day. Treatment is initiated with an IV loading dose of sotalol hydrochloride in an amount of about 60 mg over a period of about 1 hour. The patient's QTc interval is measured every 15 minutes during the IV loading dose administration, along with heart rate and blood pressure. The patient's QTc interval does not exceed 500 ms during any of these measurements, and the ΔQTc is less than 20%.

A first oral dose of 80 mg can be administered to the subject about 5 hours after initiation of the IV loading dose (or about 4 hours after completion of the IV loading dose). Patients can be administered the first and/or subsequent oral doses in a facility capable of providing cardiac resuscitation and continuous electrographic monitoring or after being released from such facility. The patient's QTc interval is monitored following administration of the first oral dose. The patient's QTc interval still does not exceed 500 ms and the patient's ΔQTc is less than 20%. The patient would achieve or be expected to achieve a steady state Cₘₐₓ within about 8 hours of administration of the IV sotalol. Monitoring could then be discontinued and subsequent oral doses of 80 mg would be administered by the patient every 12 hours.

The present disclosure has described particular implementations having various features. In light of the disclosure provided herein, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A method of administering sotalol hydrochloride therapy using a single intravenous (IV) dosage, the method comprising:
(1) administering to a subject a single IV dosage of sotalol hydrochloride, for a period of about 1 hour, wherein the single IV dosage is administered:
(a) in an amount of about 49.5-128 mg, for the subject who is naive to sotalol or who has not received sotalol for at least five (5) half-lives of sotalol;
(b) in an amount of about 73.8-105.6 mg, for the subject who had received 80 mg of oral sotalol hydrochloride prior to the IV dosage; or
(c) in an amount of about 88-141 mg, for the subject who had received 120 mg of oral sotalol hydrochloride prior to the IV dosage; or
(d) in an amount of about 140-260 mg, for the subject who had received 160 mg of oral sotalol hydrochloride prior to the IV dosage; and
(2) starting at least about 1 hour after completion of the single IV dosage, administering an oral dosage of sotalol hydrochloride selected from:
(a) 80 mg or 120 mg, such as after completion of the (1)(a) IV dosage; or
(b) 120 mg, such as after completion of the (1)(b) IV dosage;
(c) 160 mg, such as after completion of the (1)(c) IV dosage; or
(d) 240 mg, such as after completion of the (1)(d) IV dosage.

2. The method of claim 1, wherein:
the single IV dosage and the oral dosage are administered to the subject while admitted to a facility capable of providing cardiac resuscitation and continuous electrographic monitoring;
or the single IV dosage is administered to the subject while admitted to a facility capable of providing cardiac resuscitation and continuous electrographic monitoring, then the subject is released from the facility before any oral dosages of sotalol hydrochloride are administered;
optionally, wherein the oral dosage is administered about 1-4 hours after completion of the administration of the IV loading dose.

3. The method of claim 1, wherein:
the single IV dosage and the oral dosage are administered to the subject while admitted to a facility capable of providing cardiac resuscitation and continuous electrographic monitoring;
the subject is released from the facility capable of providing cardiac resuscitation and continuous electrographic monitoring; and
the subject is administered one or more additional oral dosage of sotalol hydrochloride.

4. The method of claim 1, wherein the oral dosage is administered about 1-4 hours after completion of the administration of the IV loading dose.

5. The method of claim 1, wherein the subject experiences a sotalol hydrochloride Cₘₐₓ steady state within about 8 hours of the administering of the single IV dose.

6. The method of claim 1, wherein the subject has a cardiovascular condition selected from atrial fibrillation, atrial flutter, atrial tachycardia, focal atrial tachycardia, ventricular tachycardia, hemodynamically stable or unstable ventricular tachycardia, paroxysmal atrial fibrillation, ventricular fibrillation, supraventricular tachycardia, atrioventricular re-entrant tachycardia, atrioventricular nodal re-entrant tachycardia, paroxysmal supraventricular tachycardia, junctional ectopic tachycardia, junctional tachycardia, heart failure, coronary artery disease, and pulmonary artery hypertension, or any combination thereof.

7. The method of claim 6, wherein the subject is being treated for atrial fibrillation and/or atrial flutter and is currently in normal sinus rhythm.

8. The method of claim 1, wherein:
the single IV dosage is in an amount of 49.5-128 mg;
the oral dosage is 80 mg or 120 mg; and
the subject is naive to sotalol or has not received sotalol for at least five (5) half-lives of sotalol.

9. The method of claim 1, wherein:
the single IV dosage is in an amount of 73.8-105.6 mg;
the oral dosage is 120 mg; and
the subject had received 80 mg of oral sotalol hydrochloride prior to the IV dosage.

10. The method of claim 1, further comprising:
determining a first QTc interval of the patient is less than 450 ms; and
determining a second QTc interval of the patient is less than 500 ms and is less than a 20% increase from the first QTc interval.

11. A method of initiating or escalating sotalol hydrochloride treatment in a patient, comprising:
(A) determining a creatinine clearance of the patient is >60 mL/min;
(B) determining a first QTc interval of the patient is 450 ms or less;
(C) administering to the patient an IV loading dose of sotalol hydrochloride, wherein the IV loading dose is chosen from an amount ranging from:
i. 55-88 mg, wherein the patient is being initiated for a target oral dose of 80 mg;
ii. 70-88 mg, wherein the patient is being is being escalated to a target oral dose of 120 mg;
iii. 82 mg to 140.8 mg, wherein the patient is being initiated for a target oral dose of 120 mg, or is being escalated to a target oral dose of 160 mg or 240 mg;
(D) determining a second QTc interval of the patient is less than 500 ms and is less than a 20% increase from the first QTc interval; and
(E) administering a first oral dose of the sotalol hydrochloride to the patient after completion of the administering of the IV loading dose.

12. The method of claim 11, wherein the first oral dose is administered at least about 1 hour after completion of the administration of the IV loading dose.

13. The method of claim 11, wherein the patient experiences a sotalol hydrochloride Cₘₐₓ steady state within about 8 hours of the administering of the single IV dose.

14. The method of claim 11, wherein the patient has a cardiovascular condition selected from atrial fibrillation, atrial flutter, atrial tachycardia, focal atrial tachycardia, ventricular tachycardia, hemodynamically stable or unstable ventricular tachycardia, paroxysmal atrial fibrillation, ventricular fibrillation, supraventricular tachycardia, atrioventricular re-entrant tachycardia, atrioventricular nodal re-entrant tachycardia, paroxysmal supraventricular tachycardia, junctional ectopic tachycardia, junctional tachycardia, heart failure, coronary artery disease, and pulmonary artery hypertension, or any combination thereof.

15. The method of claim 14, wherein the patient is being treated for atrial fibrillation and/or atrial flutter and is currently in normal sinus rhythm.

16. The method of claim 11, wherein:
the patient is being initiated for a target oral dose of 80 mg;
the creatinine clearance of the patient is >90 mL/min; and
the IV loading dose is administered in an amount of 55-63 mg.

17. The method of claim 11, wherein:
the patient is being initiated for a target oral dose of 120 mg;
the creatinine clearance of the patient is >90 mL/min; and
the IV loading dose is administered in an amount of 82 mg to 96 mg.

18. The method of claim 11, wherein:
the patient is being escalated to a target oral dose of 120 mg;
the creatinine clearance of the patient is >90 mL/min; and
the IV loading dose is administered in an amount of 70 mg to 80 mg.

19. The method of claim 11, wherein at least one oral dose amount is based on the first QTc interval and/or the second QTc interval.

20. The method of claim 1, wherein:
the subject has a cardiovascular condition selected from atrial fibrillation, atrial flutter, or ventricular tachycardia;
the oral dosage is administered about 1-4 hours after completion of the administration of the IV loading dose; and
there is less than a 20% increase in a change in QTc interval of the subject after administering the IV dosage.
